(19) United States
(12) Patent Application Publication
(10) Pub. No.: US 2023/0157681 A1
(43) Pub. Date: May 25, 2023

(54) MINIMALLY INVASIVE ANCHOR DRILL SYSTEMS

(71) Applicant: AEVUMED, INC., Malvern, PA (US)

(72) Inventors: Saif Khalil, Malvern, PA (US); Miles Curtis, Philadelphia, PA (US)

(21) Appl. No.: 18/058,366

(22) Filed: Nov. 23, 2022

Related U.S. Application Data
(60) Provisional application No. 63/283,016, filed on Nov. 24, 2021.

(51) Int. Cl. A61B 17/04

(52) U.S. Cl. CPC A61B 17/0401; A61B 2017/0409; A61B 2017/044; A61B 17/00234

(57) ABSTRACT

The present invention provides minimally invasive anchor drill devices for drilling pilot holes and inserting hardware into the pilot holes. The devices perform both functions without needing to be removed from a site of drilling, ensuring accurate placement of hardware while streamlining minimally invasive surgical procedures. The present invention also provides suture anchors capable of simultaneously supporting locking and re-tensioning suture configurations. The anchor drill devices and suture anchors can be used together for anchor-first procedures, suture-first procedures, and procedures linking several anchors together through combinations of locking and re-tensioning suture engagements.

### CROSS-REFERENCE TO RELATED APPLICATIONS

This application claims priority to U.S. provisional application No. 63/283,016 filed Nov. 24, 2021, incorporated herein by reference in its entirety.

### BACKGROUND OF THE INVENTION

Endoscopic surgery encompasses several minimally invasive techniques where surgical access to a subject's inner body is accessed and maintained through one or more small incisions or ports. In certain procedures, pilot holes are driven into tissue using drills for the insertion of screws or other instrumentation into the pilot holes. However, after removal of the drills, it is often difficult to locate the pilot hole or to properly align hardware with the pilot holes due to the shifting of tools and subject tissue.

Thus, there is a need in the art for improved devices for drilling and placing hardware in a minimally invasive setting. The present invention meets this need.

### SUMMARY OF THE INVENTION

In one aspect, the present invention relates to an anchor drill device comprising: a proximal handle connected to a distal guide end by a shaft; at least a first lumen and a second lumen, the first and second lumens extending through the handle and the shaft; and a third lumen extending through the guide end; wherein the first lumen and second lumens converge into the third lumen at the guide end.

In one embodiment, a drill stem terminating in a distal drill tip is positioned in the first lumen such that the distal drill tip is extendable out of the first lumen, through the third lumen, and past the guide end. In one embodiment, an anchor introducer engageable to a suture anchor at a distal end is positioned in the second lumen such that the suture anchor is extendable out of the second lumen, through the third lumen, and past the guide end. In one embodiment, the guide end comprises a pointed or serrated tip. In one embodiment, the guide end is angled relative to a longitudinal axis of the handle and shaft by an angle between about 0° and 90°. In one embodiment, the first lumen and the second lumen are stacked in a direction of the guide end angle. In one embodiment, the first lumen, the second lumen, and the third lumen each comprise a plurality of apertures. In one embodiment, a suture slot extends from the guide end to a position on the shaft. In one embodiment, the first lumen and/or the second lumen comprises a spring-loaded button.

In one aspect, the present invention relates to a suture anchor device, comprising: an anchor body; a suture port formed laterally in the anchor body; a suture post positioned across the suture port; and an introducer port positioned at a proximal end of the anchor body; wherein the introducer port is connected to the suture port by a lumen running longitudinally through the anchor body.

In one embodiment, the suture port is configured to support knotless locking suture engagements and re-tensioning suture engagements. In one embodiment, the suture port is positioned near a distal end of the anchor body. In one embodiment, the suture port is positioned near a proximal end of the anchor body and a second suture port is positioned near a distal end of the anchor body. In one embodiment, the suture port positioned near the proximal end of the anchor body is configured to support re-tensioning suture engagements, and the second suture port is configured to support knotless locking suture engagements. In one embodiment, the suture port positioned near the proximal end of the anchor body faces a direction oriented about 90° relative to a direction of the second suture port.

In one aspect, the present invention relates to a suture anchor device, comprising: an anchor body; a proximal suture port formed laterally in the anchor body; a distal suture port formed laterally in the anchor body; and a suture post at a distal end of the anchor body, the suture post being positioned across the proximal suture port.

In one embodiment, the first suture port is configured to support re-tensioning suture engagements and the second suture port is configured to support knotless locking suture engagements.

In one aspect, the present invention relates to a method of repairing tissue, the method comprising the steps of: providing an anchor drill device, the anchor drill device comprising at least a first and a second lumen extending through a shaft towards a shaft distal end, wherein the first and second lumens converge into a third lumen extending out of the shaft distal end; pressing the shaft distal end against a tissue site; extending a first instrument through the first lumen and the third lumen; performing a first procedure step at the tissue site; retracting the first instrument from the shaft distal end while maintaining the shaft distal end against the tissue site; extending at least one second instrument through the second lumen and the third lumen; and performing at least one second procedure step at the tissue site.

In one embodiment, the tissue site is a labral tear. In one embodiment, the first instrument is a drill stem comprising a distal drill tip. In one embodiment, the first procedure step is drilling a pilot hole. In one embodiment, the at least one second instrument is an anchor introducer comprising a distal suture anchor. In one embodiment, the at least one second procedure step is driving a suture anchor into a pilot hole. In one embodiment, the suture anchor comprises a suture port configured to support re-tensioning suture engagements and a suture port configured to support knotless locking suture engagements. In one embodiment, the method steps are repeated at least one time, such that a plurality of suture anchors is driven around a labral tear. In one embodiment, the plurality of suture anchors is linked together by at least one suture thread in a continuous chain.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a perspective view of an exemplary anchor drill device.

FIG. 2 depicts a side view of a handle of an exemplary anchor drill device.

FIG. 3 depicts a magnified view of a distal end of an exemplary anchor drill device, wherein a suture slot is visible.

FIG. 4 depicts a magnified view of a distal end of an exemplary anchor drill device (top), and a side cross-sectional view of a shaft and distal end of an exemplary anchor drill device (bottom), wherein a first and second lumen are shown using dashed lines.

FIG. 5 depicts a perspective view of an exemplary anchor drill device provided with a drill stem and an anchor introducer.

FIG. 6 depicts a magnified view of a distal end of an exemplary anchor drill device, wherein a drill tip and an anchor are each in a retracted position.

FIG. 7 depicts a magnified view of a distal end of an exemplary anchor drill device, wherein a drill tip is in an extended position and an anchor is in a retracted position.

FIG. 8 depicts a magnified view of a distal end of an exemplary anchor drill device, wherein a drill tip is in a retracted position and an anchor is in an extended position.

FIG. 9 depicts a magnified view of a proximal end (top) and a distal end (bottom) of an exemplary anchor drill device, wherein an additional instrument (suture retriever) is provided in a lumen of the device.

FIG. 10 depicts perspective views of an exemplary suture anchor with (bottom left) and without (top right) a suture engaged thereto.

FIG. 11 depicts a perspective view of an exemplary suture anchor.

FIG. 12 depicts a perspective view of an exemplary suture anchor engaged to an anchor introducer (top) and disengaged from an anchor introducer (bottom).

FIG. 13 is a flowchart of an exemplary method of repairing tissue.

FIG. 14 is a schematic illustrating an exemplary method of anchor-first insertion.

FIG. 15 is a schematic illustrating an exemplary method of suture-first insertion.

FIG. 16 is a schematic illustrating an exemplary method of continuous chain suture anchor insertion (left) and continuous chain suture anchor repair of a labral tear (right).

### DETAILED DESCRIPTION

The present invention provides minimally invasive systems comprising anchor drill devices and suture anchors. The anchor drill devices are configured to drill pilot holes and insert hardware into the pilot holes without needing to be removed from a site of drilling, ensuring accurate placement of hardware while streamlining minimally invasive surgical procedures. The suture anchors simultaneously support locking and re-tensioning suture configurations. The anchor drill devices and suture anchors can be used together for anchor-first procedures, suture-first procedures, and procedures linking several anchors together through combinations of locking and re-tensioning suture engagements.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Minimally Invasive Anchor Drill Systems

The present invention provides in part minimally invasive surgical systems comprising anchor drill devices and suture anchors. The anchor drill devices are configured to maintain a position on a tissue surface for the entire duration of a minimally invasive surgical procedure, such that drilling and placement of hardware at the tissue surface is carried out with consistent positioning. The suture anchors are configured to support knotless locking suture engagements, re-tensioning suture engagements, and both simultaneously. The minimally invasive systems are suitable for use in any surgical procedure, including but not limited to arthroscopy, laparoscopy, thoracoscopy, and the like. In some embodiments, the systems are suitable for minimally invasive joint repair, tendon repair, and ligament repair. While the systems are described as comprising anchor drill devices and suture anchors, it should be understood that the devices can be used separately or in combination with additional instrumentation.

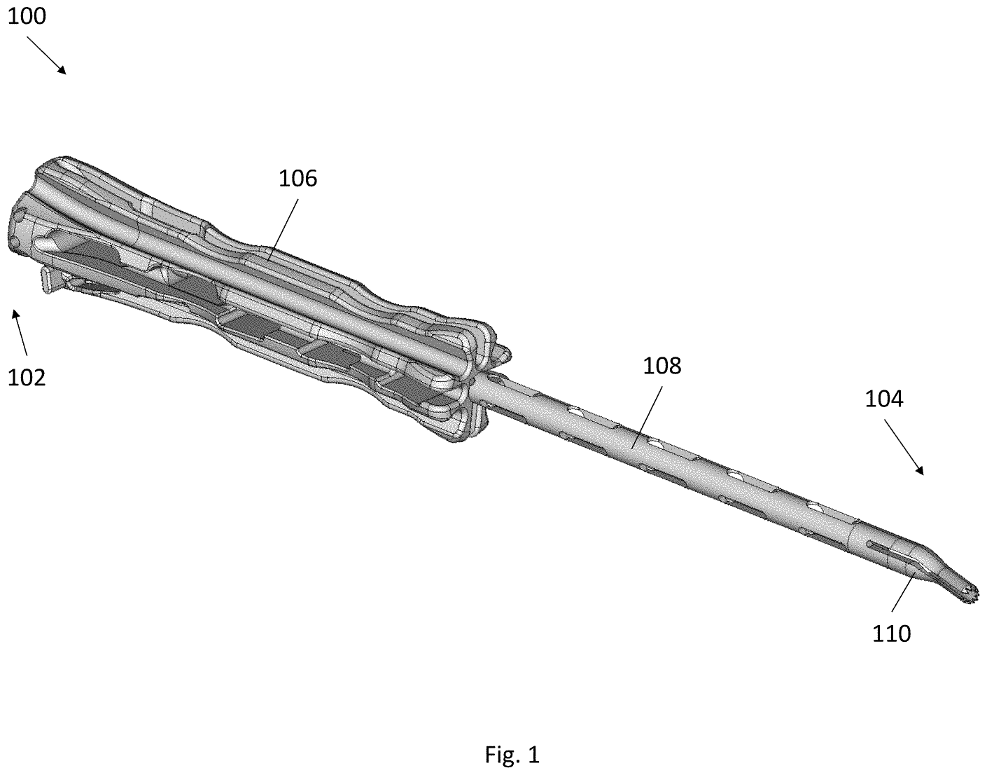

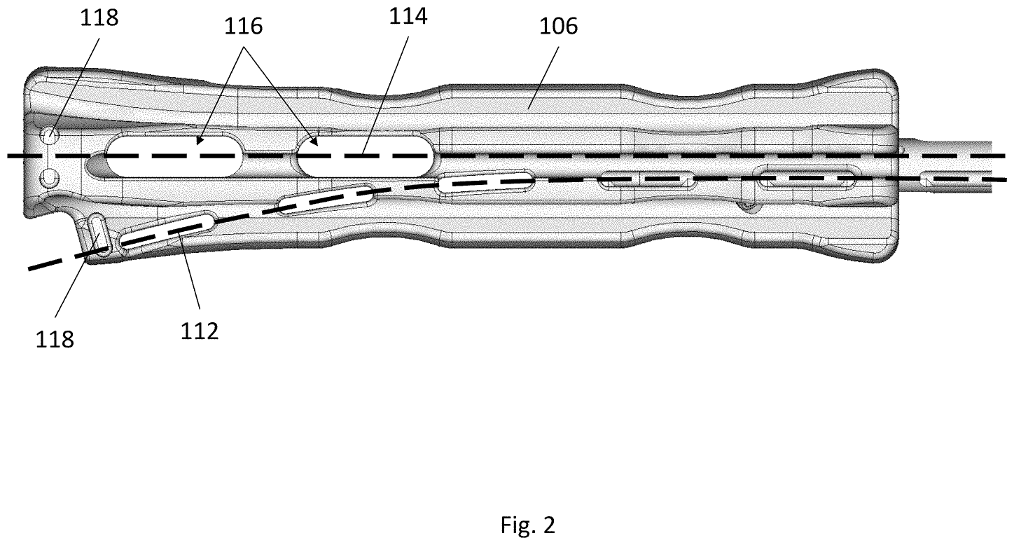

Referring now to FIG. 1, an exemplary anchor drill device 100 is now described. Device 100 comprises a handle 106 at proximal end 102, a guide end 110 at a distal end 104, and a shaft 108 connecting handle 106 to guide end 110. FIG. 2 depicts a side view of handle 106, wherein dashed lines are shown tracing paths for a first lumen 112 and a second lumen 114. Each of the lumens 112 and 114 extend from proximal openings in handle 106, through shaft 108, and into guide 110. While device 100 is depicted having two lumens, it should be understood that device 100 can have any desired number of lumens, including a third lumen, a fourth lumen, a fifth lumen, and the like. In some embodiments, each of the lumens comprises a plurality of apertures 116. Apertures 116 enable visualization of instrumentation positioned within each of the lumens. In some embodiments apertures 116 facilitate access to each of the lumens for ease of cleaning and disinfecting, such as by autoclave. In some embodiments, handle 106 comprises one or more buttons 118. Buttons 118 can be spring-loaded or toggled to at least partially constrict a location of a lumen, such that instrumentation positioned within a lumen may be temporarily locked in place or actuate within a lumen in a stepped manner.

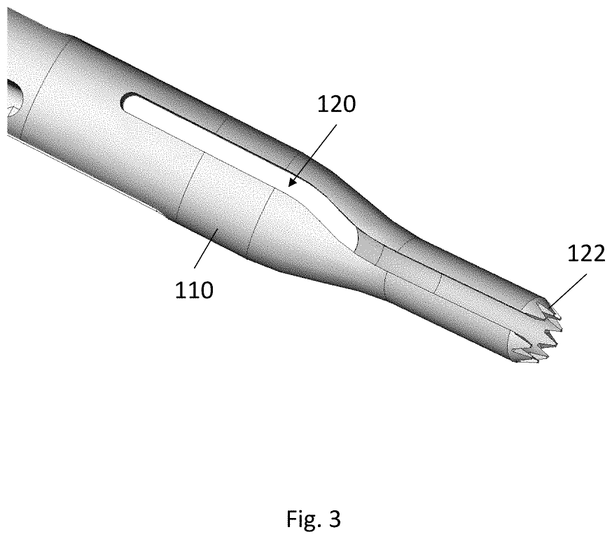

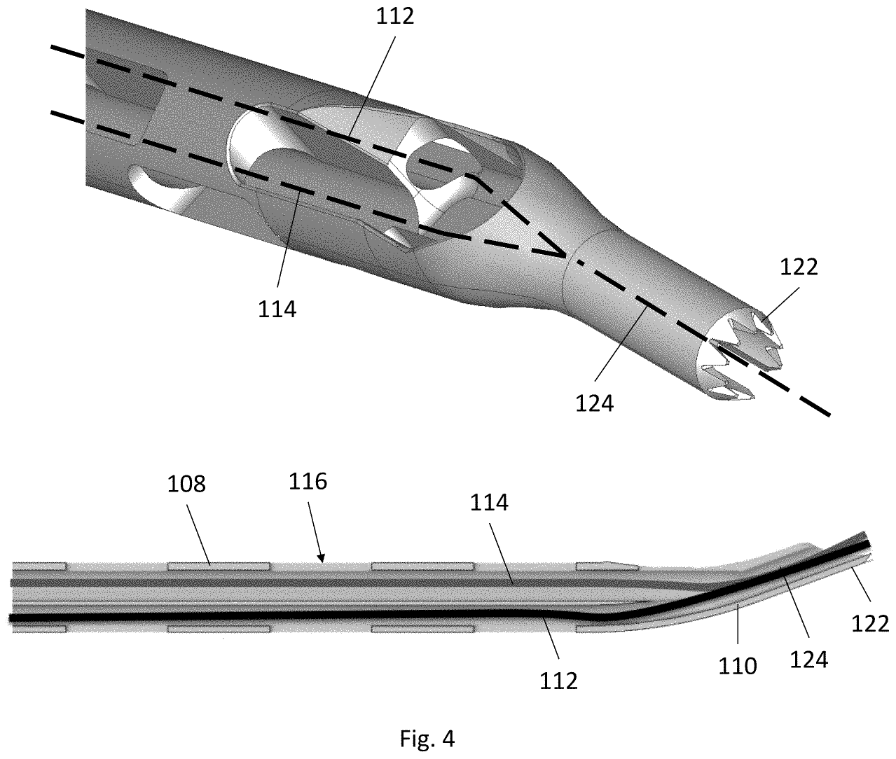

Referring now to FIG. 3 and FIG. 4, guide end 110 is now described. Guide end 110 comprises a suture slot 120 that extends towards a guide tip 122. In some embodiments, guide tip 122 comprises a pointed or serrated end configured to enhance grip on a tissue. In FIG. 4, lumens 112 and 114 (depicted by dashed lines) are shown converging into guide end lumen 124 (depicted by dashed lines). Accordingly, instrumentation positioned within any of the lumens of handle 106 is configured to extend through shaft 108 and out of guide end lumen 124 of guide end 110. In some embodiments, guide end 110 is positioned at an angle relative to a longitudinal axis of handle 106 and shaft 108. The angle can be any desired angle between about 0° and 90°, or between about 15° and 45°. In various embodiments, the lumens of device 100 can be oriented in any desired arrangement within shaft 108. Lumen arrangement in shaft 108 relative to guide end lumen 124 may be modified to accommodate instruments inserted therein. For example, flexible and/or thin instruments may tolerate sharper bends and more bending points, while less flexible and/or thick instruments may be suited to lesser bends and fewer bending points. In one embodiment, device 100 comprises a lumen 112 stacked on a lumen 114, such that the stacked lumens are in alignment with a curvature of guide end 110.

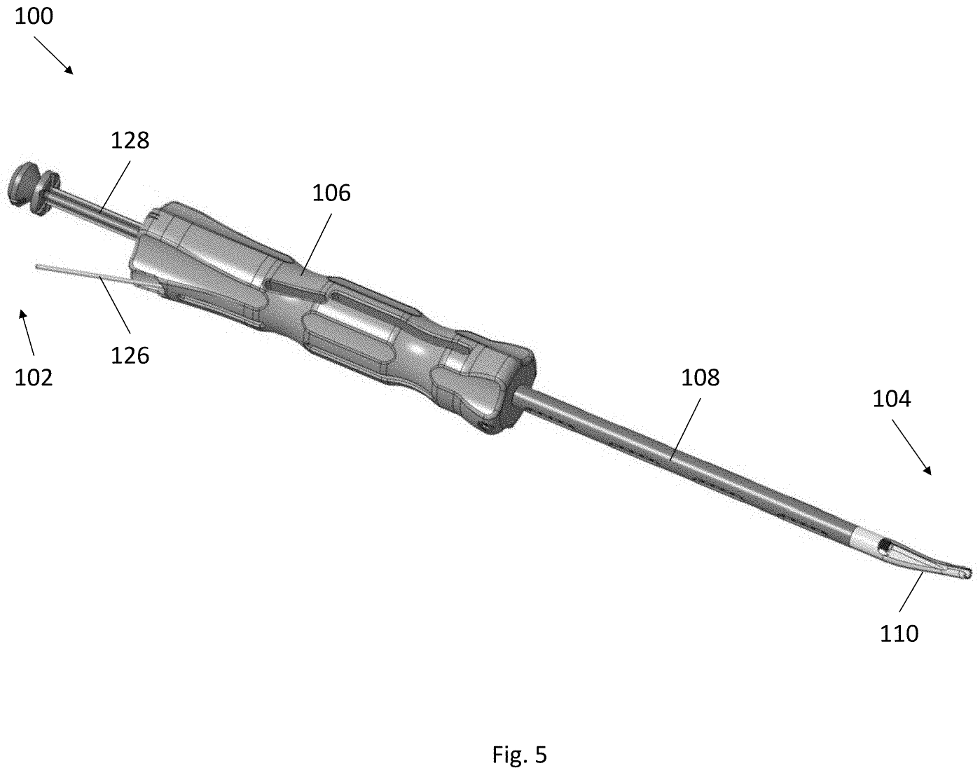

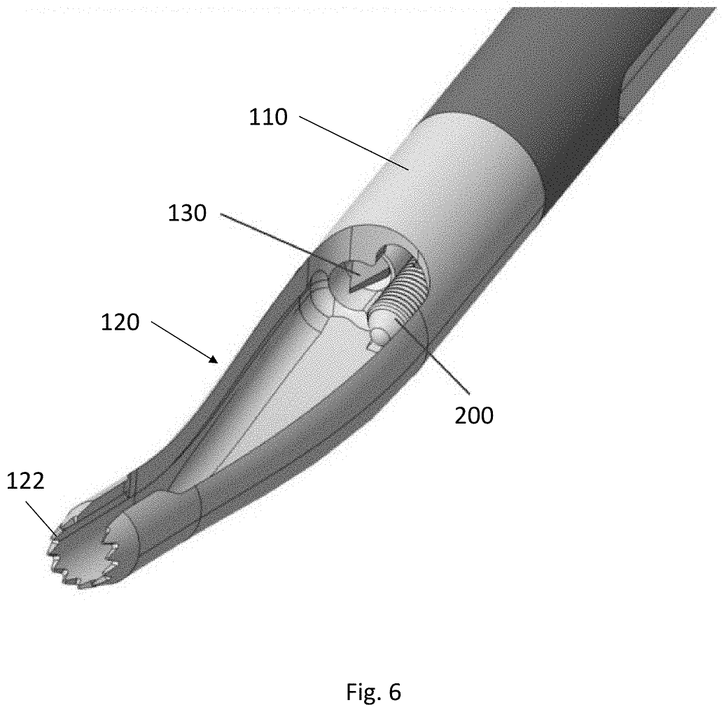

FIG. 5 through FIG. 9 illustrate exemplary functionality of device 100 described above. FIG. 5 depicts a drill stem 126 positioned within lumen 112 and an anchor introducer 128 positioned within lumen 114. The distal ends of drill stem 126 and anchor introducer 128 are visible at guide end 110 in FIG. 6, wherein drill tip 130 and anchor 200 are positioned at distal openings of lumen 112 and 114, respectively. A surgical procedure may be initiated by pressing guide end 110 against a tissue surface, such that guide tip 122 securely grips the tissue surface ensuring little to no movement between guide tip 122 and the tissue surface for the duration of the surgical procedure.

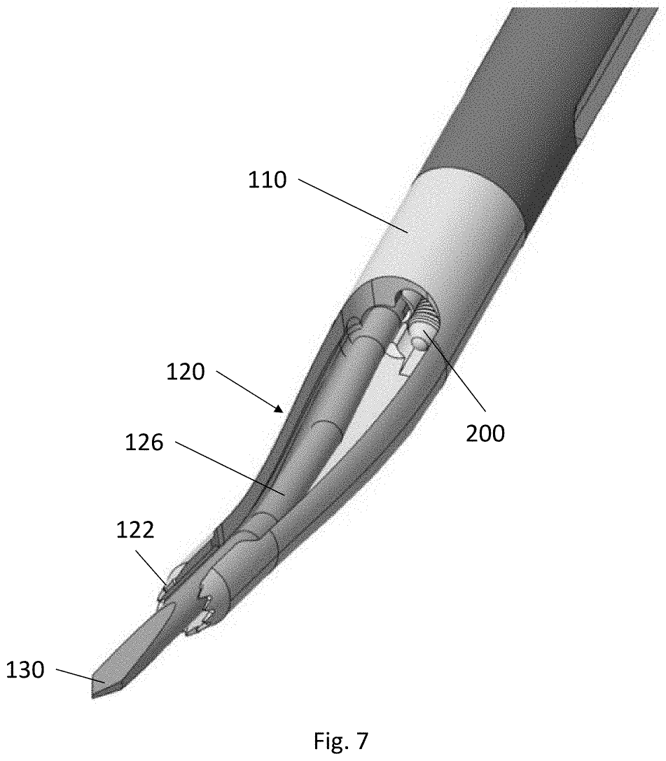

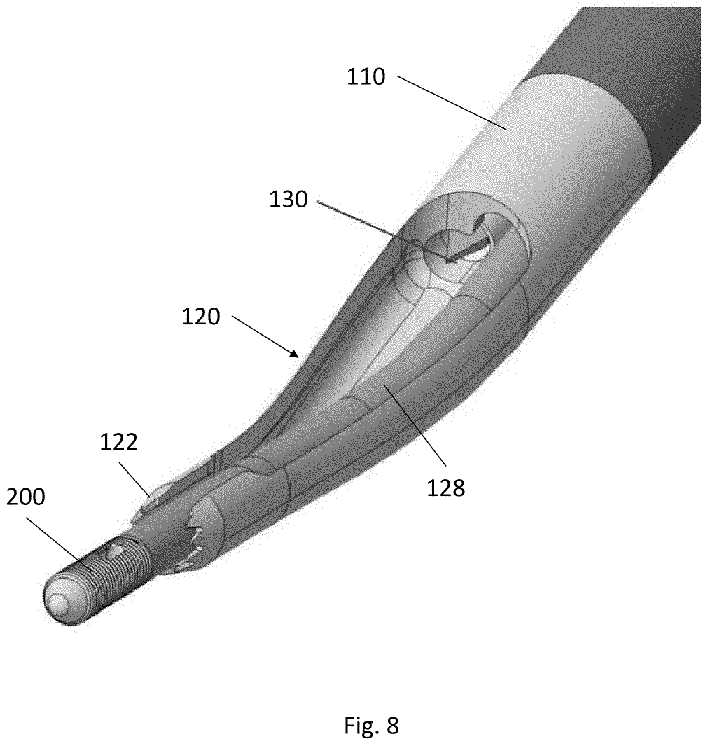

In FIG. 7, drill stem 126 is extended out of lumen 112 such that drill tip 130 is guided through guide end lumen 124 and out of guide tip 122. As would be understood by persons having skill in the art, drill stem 126 may be actuated to drill into the gripped tissue surface with drill tip 130 to form a pilot hole having a depth substantially equivalent to a length of drill tip 130 extending past guide tip 122. In FIG. 8, drill stem 126 and drill tip 130 are retracted into lumen 112 and anchor introducer 128 is extended out of lumen 114 such that anchor 200 is guided through guide end lumen 124 and out of guide tip 122. As described above, guide tip 122 grips the tissue surface to prevent movement throughout the procedure such that guide tip 122 remains positioned over the pilot hole formed by drill tip 130. As would be understood by persons having skill in the art, anchor 200 is accurately guided into the pilot hole by guiding anchor 200 through guide tip 122. In various embodiments, anchor 200 may be driven or hammered into the pilot hole, whereupon anchor introducer 128 may be retracted to leave anchor 200 in the tissue.

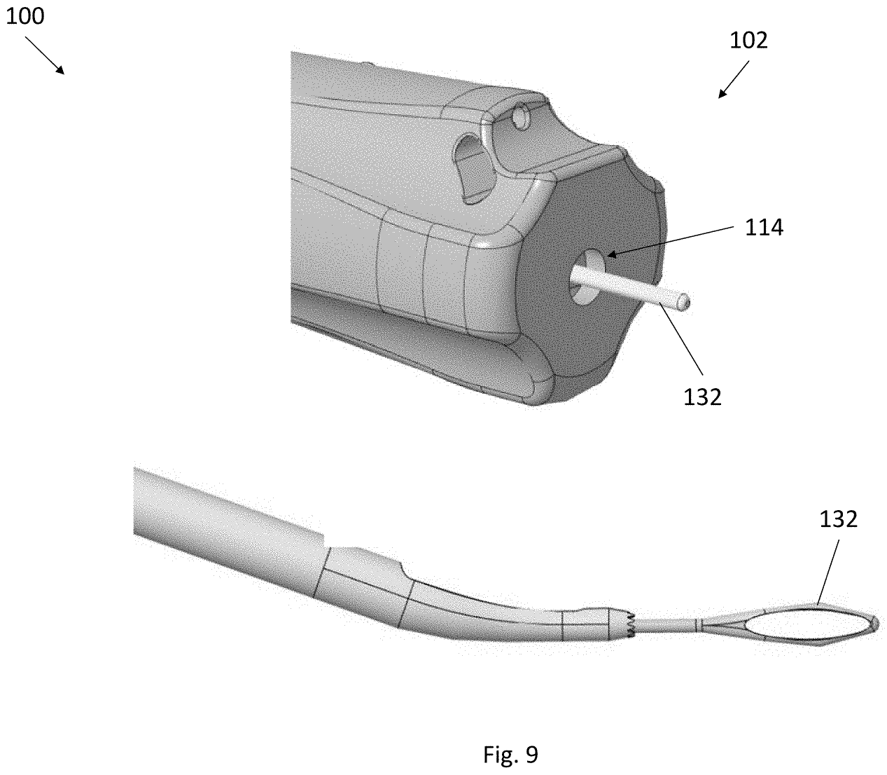

In various embodiments, one or more additional procedure steps may be conducted while maintaining device 100 in position over the pilot hole and/or anchor 200. For example, FIG. 9 depicts a suture retriever 132 inserted into lumen 114 such that lumen 114 guides suture retriever 132 to an inserted anchor 200. It should be understood that any suitable instrument may be inserted into a lumen of device 100 to perform one or more procedure steps at a pilot hole or inserted anchor 200 site.

Contemplated anchor drill devices described herein can have any suitable length, shape, and diameter. For example, contemplated anchor drill devices can have a shaft length between about 5 cm to about 50 cm and a shaft outer diameter between about 5 mm to about 25 mm. Anchor drill device handles can comprise any desired grip shape and exterior texture. In various embodiments, handles can comprise one or more notches configured to temporarily hold a suture thread.

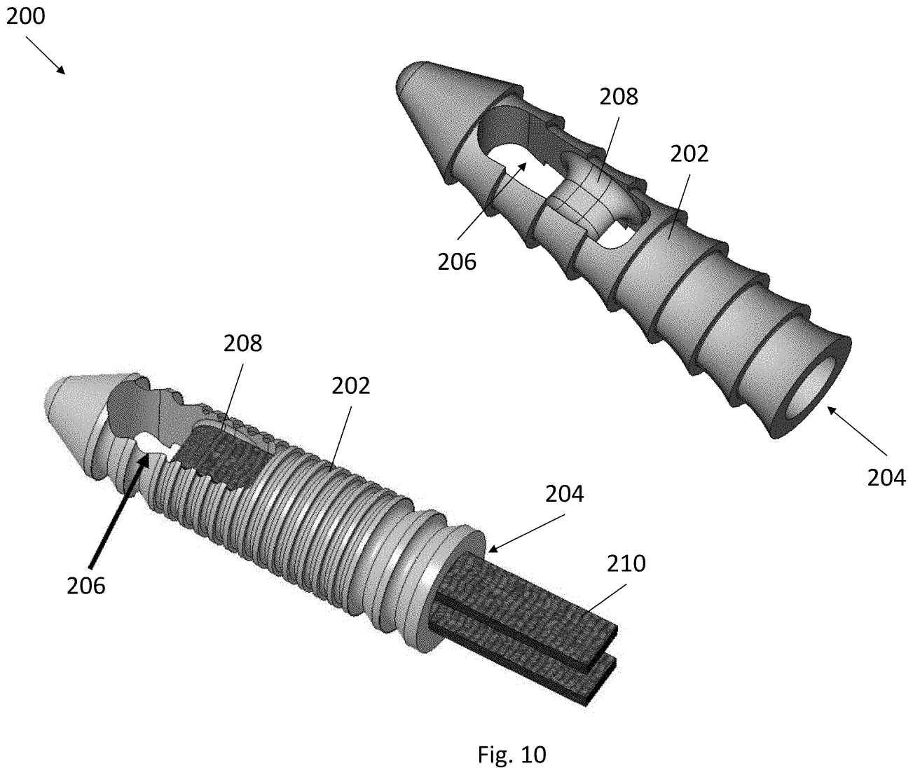

Referring now to FIG. 10 through FIG. 12, exemplary suture anchors are now described. FIG. 10 depicts a suture anchor 200 comprising an anchor body 202, an introducer port 204, a suture port 206, and a suture post 208 positioned across suture port 206. Introducer port 204 is configured to engage to a distal end of an anchor introducer 128, as described elsewhere herein. In some embodiments, introducer port 204 comprises a polygonal or non-circular shape configured to resist rotational movement when engaged to an anchor introducer 128. Introducer port 204 is connected to suture port 206 by a lumen running longitudinally through anchor body 202, such that one or more suture threads 210 are passable through introducer port 204 to loop around suture post 208 (bottom left of FIG. 10). In some embodiments, suture port 206 is positioned adjacent to or near a distal end of anchor 200. Suture port 206 supports knotless locking suture engagement and re-tensioning suture engagement. For example, the depicted suture anchor 200 is shown in a re-tensioning suture engagement, such that the engaged suture thread moves freely about suture post 208 when anchor 200 is driven into tissue. In a knotless locking suture engagement, a suture thread may be routed directly through suture port 206 such that driving suture anchor 200 into tissue locks a length of the suture thread between the tissue and an exterior of anchor body 202. Suture anchor 200 is configured to receive both a suture through introducer port 204 and directly through suture port 206 to simultaneously engage a knotless locking suture thread and a re-tensioning suture thread.

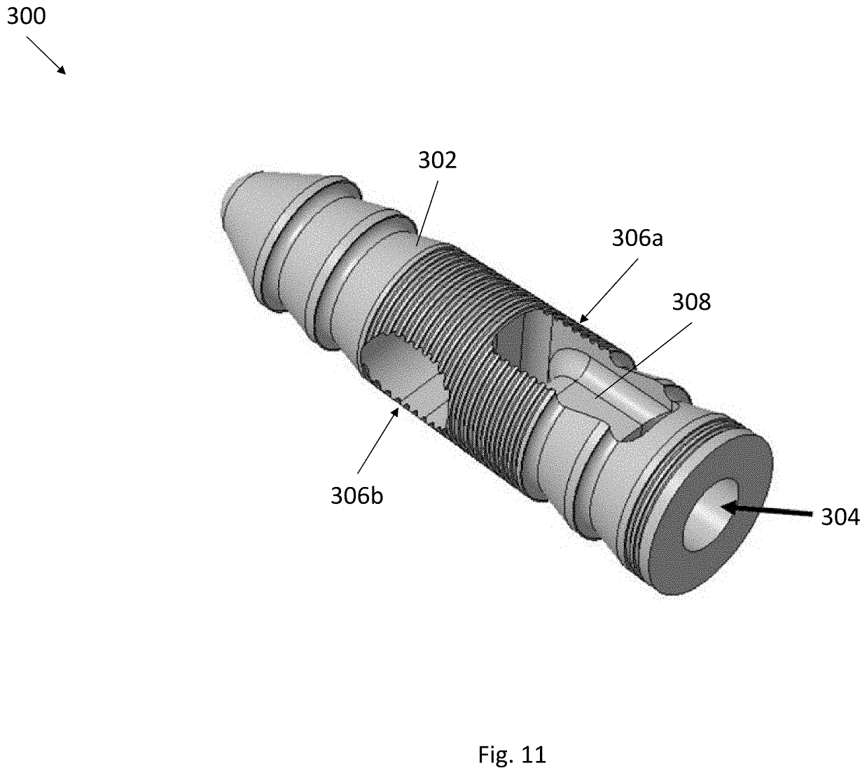

FIG. 11 depicts a suture anchor 300 comprising an anchor body 302, an introducer port 304, a proximal re-tensioning suture port 306a, a distal knotless locking suture port 306b, and a suture post 308 positioned across re-tensioning suture port 306a. Introducer port 304 is configured to engage to a distal end of an anchor introducer 128, as described elsewhere herein. In some embodiments, introducer port 304 comprises a polygonal or non-circular shape configured to resist rotational movement when engaged to an anchor introducer 128. Introducer port 304 is connected to re-tensioning suture port 306a by a lumen, such that one or more suture threads are passable through introducer port 304 to loop around suture post 308. In a re-tensioning suture engagement, an engaged suture thread moves freely about suture post 308 when anchor 300 is driven into tissue. In a knotless locking suture engagement, a suture thread may be routed directly through knotless locking suture port 306b such that driving suture anchor 300 into tissue locks a length of the suture thread between the tissue and an exterior of anchor body 302. Suture anchor 300 is configured to receive both a suture through introducer port 304 and directly through knotless locking suture port 306b to simultaneously engage a knotless locking suture thread and a re-tensioning suture thread. In some embodiments, a suture thread may be routed directly through re-tensioning suture port 306a similarly to knotless locking suture port 306b to also function as knotless locking suture port. Re-tensioning suture port 306a and knotless locking suture port 306b are depicted as being oriented rotationally about 90° to each other relative to anchor body 302. However, it should be understood that any desired orientation is contemplated.

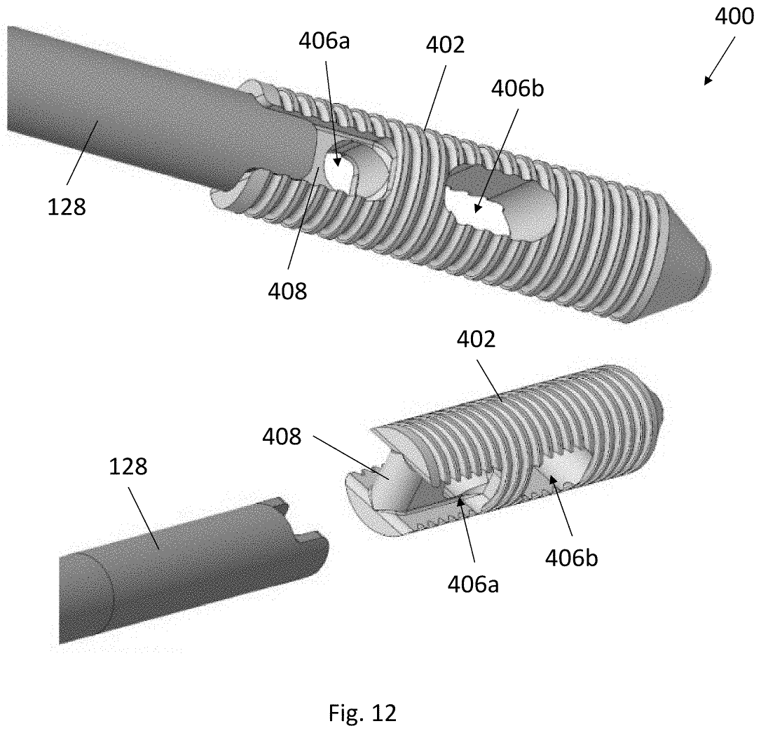

FIG. 12 depicts a suture anchor 400 comprising an anchor body 402, a proximal re-tensioning suture port 406a, a distal knotless locking suture port 406b, and a suture post 408 positioned across re-tensioning suture port 406a. A distal portion of anchor body 402 and suture post 408 forms an engagement structure configured to mate with a distal end of an anchor introducer 128, wherein the engagement structure resists rotational movement with anchor introducer 128. In a re-tensioning suture engagement, an engaged suture thread is looped around and moves freely about suture post 408 when anchor 400 is driven into tissue. In a knotless locking suture engagement, a suture thread may be routed directly through knotless locking suture port 406b such that driving suture anchor 400 into tissue locks a length of the suture thread between the tissue and an exterior of anchor body 402. Suture anchor 400 is configured to receive both a suture around suture post 408 and directly through knotless locking suture port 406b to simultaneously engage a knotless locking suture thread and a re-tensioning suture thread. Re-tensioning suture port 406a and knotless locking suture port 406b are depicted as being oriented rotationally in line to each other relative to anchor body 402. However, it should be understood that any desired orientation is contemplated.

Contemplated suture anchors described herein can have any suitable length, shape, and diameter. For example, contemplated suture anchors can have a length between about 5 mm to about 50 mm and a diameter between about 2 mm to about 20 mm. Suture anchor exteriors can comprise any desired structural features configured to securely engage with a tissue substrate, such as ribbing, barbs, major threads, minor threads, and the like.

The anchor drill devices and suture anchors of the present invention can be made from any suitable material, including but not limited to metals, non-biodegradable polymers, biodegradable polymers, polymer composites, copolymers, and bioceramics. The term biodegradable as used herein is defined to mean materials that degrade in the body and then are either absorbed into or excreted from the body. The term bioceramic as defined herein is defined to mean ceramic and glass materials that are compatible with body tissue and can be biodegradable or non-biodegradable. Contemplated metals include stainless steel, titanium, alloys of nickel and titanium, or other biocompatible metallic materials.

Non-biodegradable polymers include but are not limited to polyethylene, polypropylene, PEEK (polyetheretherketone), or other biocompatible non-absorbable polymers. Biodegradable polymers include but are not limited to aliphatic polyesters, polyorthoesters, polyanhydrides, polycarbonates, polyurethanes, polyamides and polyalkylene oxides. In some embodiments, the biodegradable polymers are aliphatic polyester polymers and copolymers, and blends thereof. The aliphatic polyesters are typically synthesized in a ring opening polymerization. Suitable monomers include but are not limited to lactic acid, lactide (including L-, D-, meso and D,L mixtures), glycolic acid, glycolide, epsilon, caprolactone, p-dioxanone (1,4-dioxan-2-one), trimethylene carbonate (1,3-dioxan-2-one), delta.-valerolactone, and combinations thereof.

Bioceramics include but are not limited to ceramics comprising mono-, di-, tri-, alpha-tri-, beta-tri-, and tetra-calcium phosphate, hydroxyapatite, calcium sulfates, calcium oxides, calcium carbonates, magnesium calcium phosphates. In addition to bioceramics, bioglasses may also be used. The bioglasses may include phosphate glasses and bioglasses.

Additional contemplated polymers can include poly(amino acids), copoly(ether-esters), polyalkylene oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polyurethanes, poly(ether urethanes), poly(ester urethanes), polypropylene fumarate), poly(hydroxyalkanoate), homopolymers and copolymers of lactide (which includes lactic acid, D-,L- and meso lactide); glycolide (including glycolic acid); .epsilon-caprolactone; p-dioxanone (1,4-dioxan-2-one); trimethylene carbonate (1,3-dioxan-2-one); alkyl derivatives of trimethylene carbonate; .delta.-valerolactone; .beta.-butyrolactone; .gamma.-butyrolactone; .epsilon.-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; .alpha.,.alpha. diethylpropiolactone; ethylene carbonate; ethylene oxalate; 3-methyl-1,4-dioxane-2,5-dione; 3,3-diethyl-1,4-dioxan-2,5-dione-; 6,6-dimethyl-dioxepan-2-one; 6,8-dioxabicycloctane-7-one and polymer blends thereof. Additional exemplary polymer or polymer blends include, by non-limiting example, a polydioxanone, a polyhydroxybutyrate-co-hydrox-yvalerate, polyorthocarbonate, a polyaminocarbonate, and a polytrimethylene carbonate.

### Methods of Tissue Repair

The present invention also provides methods of tissue repair using the anchor drill devices and suture anchors described herein. In various embodiments, the tissue can be cartilage. In some embodiments, the methods are useful in repairing cartilage tears, such as a superior labrum anterior posterior (SLAP) tear.

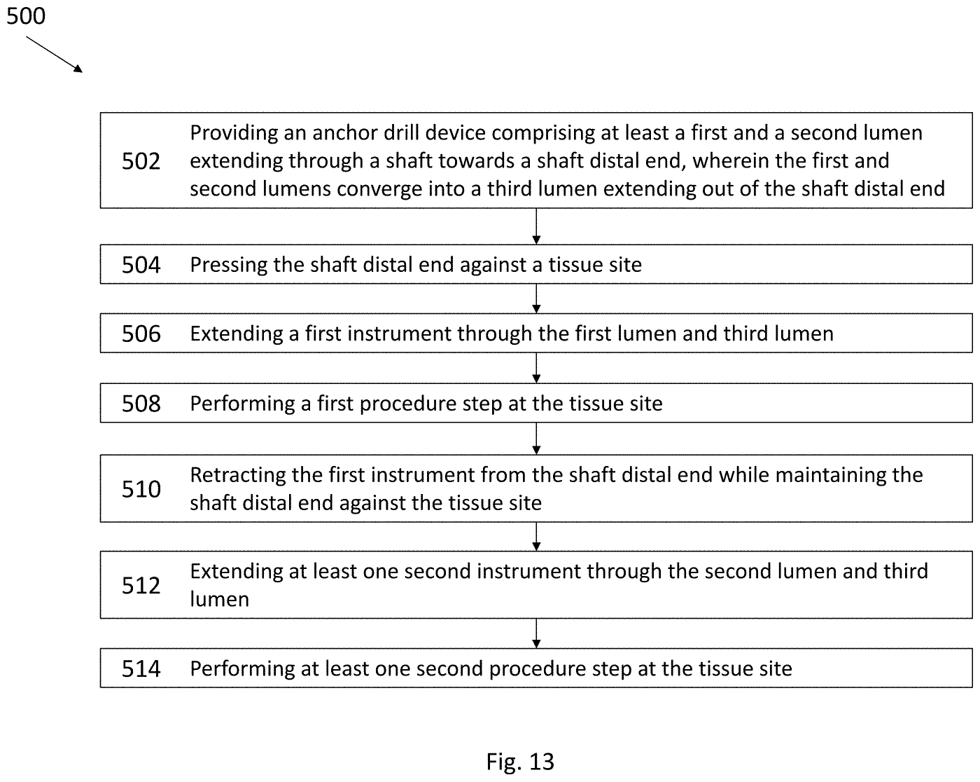

Referring now to FIG. 13, an exemplary method 500 of repairing tissue is depicted. Method 500 begins with step 502, wherein an anchor drill device is provided, the anchor drill device comprising at least a first and a second lumen extending through a shaft towards a shaft distal end, wherein the first and second lumens converge into a third lumen extending out of the shaft distal end. In step 504, the shaft distal end is pressed against a tissue site. In step 506, a first instrument is extended through the first lumen and the third lumen. In step 508, a first procedure step is performed at the tissue site. In step 510, the first instrument is retracted from the shaft distal end while maintaining the shaft distal end against the tissue site. In step 512, at least one second instrument is extended through the second lumen and the third lumen. In step 514, at least one second procedure step is performed at the tissue site.

In some embodiments, the first instrument is a drill stem comprising a drill tip, such that the first procedure step is a step of drilling a pilot hole at the tissue site. In some embodiments, the at least one second instrument is an anchor introducer comprising a suture anchor, such that the at least one second procedure step is a step of driving the suture anchor into the pilot hole. In some embodiments, the at least one second instrument is a suture retriever, such that the at least one second procedure step is a step of retrieving one or more suture threads from the tissue site.

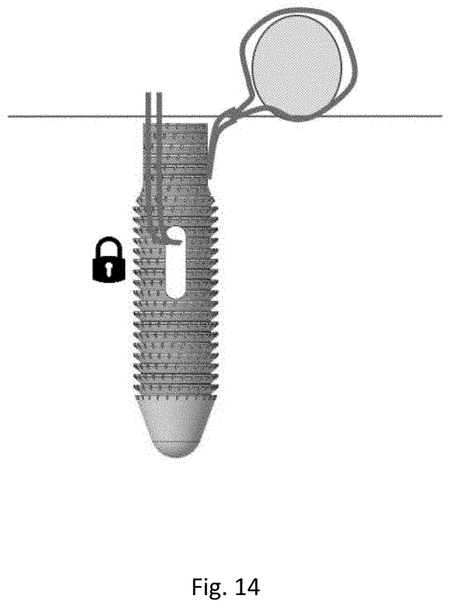

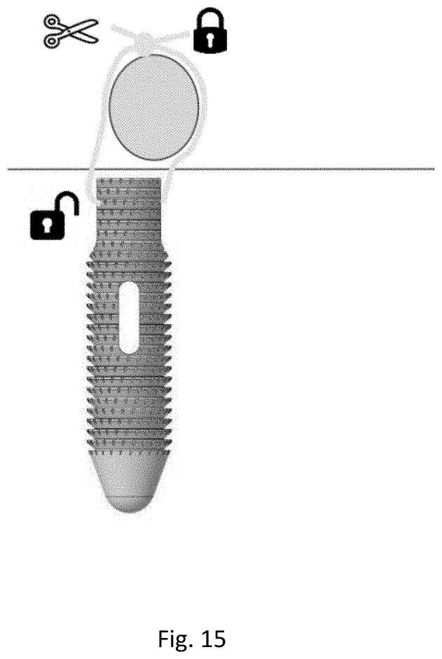

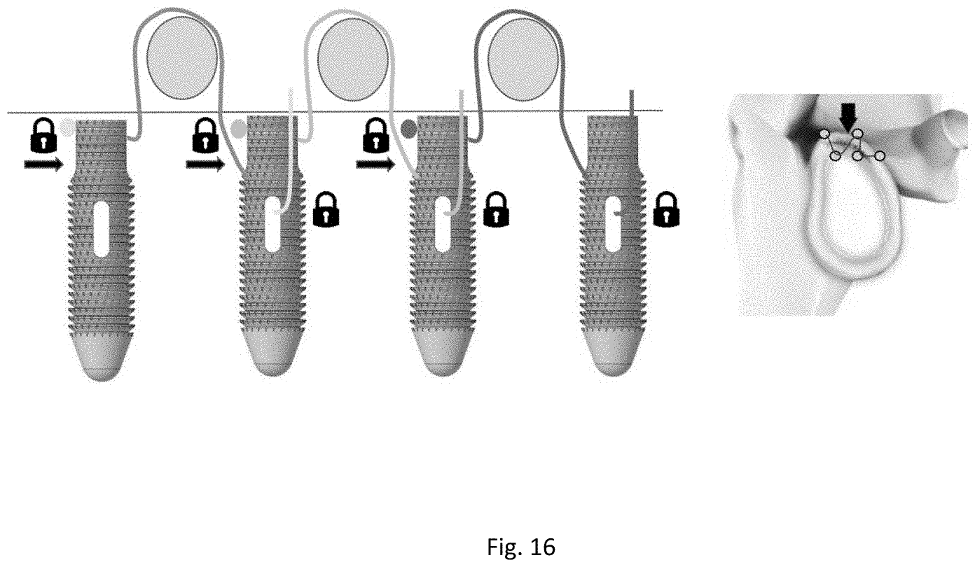

The methods can be adapted to install suture anchors into a tissue site in a variety of configurations. For example, FIG. 14 depicts an anchor-first configuration, wherein a suture anchor is driven into a tissue site with a suture thread in a re-tensioning engagement. In an anchor-first configuration, the suture anchor is driven into tissue before an adjacent tissue (depicted as an oval) is secured. The re-tensioning engagement permits the suture thread to be manipulated around the adjacent tissue and secured to the suture anchor. In another example, FIG. 15 depicts a suture-first configuration, wherein a length of suture thread is secured to an adjacent tissue first (depicted as an oval). The suture thread is then passed through a knotless locking port of a suture anchor, followed by driving the suture anchor into tissue to knotlessly lock the suture thread and adjacent tissue to the suture anchor. In another example, FIG. 16 depicts a continuous chain configuration, wherein suture anchors comprising knotless locking ports and re-tensioning ports are chained together with one or more suture threads. A first suture anchor is driven into a tissue in an anchor-first or suture-first configuration. A free suture thread from the first suture anchor is secured to an adjacent tissue (depicted as ovals), then engaged to a second suture anchor in an anchor-first or suture-first configuration. The continuous chain configuration can comprise a plurality of suture anchors linked together by one or more suture threads to secure one or more adjacent tissues. In some embodiments, the continuous chain configuration is used to close and repair a tissue tear, such as a labral tear (FIG. 16, right).

### Kits

The present invention also provides kits for tissue repair. The kits include the minimally invasive systems comprising anchor drill devices and suture anchors described elsewhere herein, as well as relevant instrumentation. For example, in some embodiments, the kits can include drills, drill bits, guide wires, suture threads, suture retrievers, and the like.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An anchor drill device comprising:
a proximal handle connected to a distal guide end by a shaft;
at least a first lumen and a second lumen, the first and second lumens extending through the handle and the shaft; and
a third lumen extending through the guide end;
wherein the first lumen and second lumens converge into the third lumen at the guide end.

2. The device of claim 1, wherein a drill stem terminating in a distal drill tip is positioned in the first lumen such that the distal drill tip is extendable out of the first lumen, through the third lumen, and past the guide end.

3. The device of claim 1, wherein an anchor introducer engageable to a suture anchor at a distal end is positioned in the second lumen such that the suture anchor is extendable out of the second lumen, through the third lumen, and past the guide end.

4. The device of claim 1, wherein the guide end comprises a pointed or serrated tip.

5. The device of claim 1, wherein the guide end is angled relative to a longitudinal axis of the handle and shaft by an angle between about 0° and 90°.

6. The device of claim 5, wherein the first lumen and the second lumen are stacked in a direction of the guide end angle.

7. The device of claim 1, wherein the first lumen, the second lumen, and the third lumen each comprise a plurality of apertures.

8. The device of claim 1, wherein a suture slot extends from the guide end to a position on the shaft.

9. The device of claim 1, wherein the first lumen and/or the second lumen comprises a spring-loaded button.

10. A suture anchor device, comprising:
an anchor body;
a suture port formed laterally in the anchor body;
a suture post positioned across the suture port; and
an introducer port positioned at a proximal end of the anchor body;
wherein the introducer port is connected to the suture port by a lumen running longitudinally through the anchor body.

11. The device of claim 10, wherein the suture port is configured to support knotless locking suture engagements and re-tensioning suture engagements.

12. The device of claim 10, wherein the suture port is positioned near a distal end of the anchor body.

13. The device of claim 10, wherein the suture port is positioned near a proximal end of the anchor body and a second suture port is positioned near a distal end of the anchor body.

14. The device of claim 13, wherein the suture port positioned near the proximal end of the anchor body is configured to support re-tensioning suture engagements, and the second suture port is configured to support knotless locking suture engagements.

15. The device of claim 13, wherein the suture port positioned near the proximal end of the anchor body faces a direction oriented about 90° relative to a direction of the second suture port.

16. A suture anchor device, comprising:
an anchor body;
a proximal suture port formed laterally in the anchor body;
a distal suture port formed laterally in the anchor body; and
a suture post at a distal end of the anchor body, the suture post being positioned across the proximal suture port.

17. The device of claim 16, wherein the first suture port is configured to support re-tensioning suture engagements and the second suture port is configured to support knotless locking suture engagements.

18-26. (canceled)
